(19) United States
(12) Patent Application Publication
(10) Pub. No.: US 2022/0059222 A1
(43) Pub. Date: Feb. 24, 2022

(54) COMPUTER-IMPLEMENTED METHOD AND COMPUTING DEVICE FOR PREDICTING CANCER

(71) Applicant: TAIPEI MEDICAL UNIVERSITY, Taipei City (TW)

(72) Inventors: Yu-Chuan LI, Taipei City (TW); Hsuan-Chia YANG, Taipei City (TW); Chih-Wei HUANG, Taipei City (TW); Phung Anh NGUYEN, Taipei City (TW); Chia-Wei LIANG, Taipei City (TW)

(21) Appl. No.: 17/001,463

(22) Filed: Aug. 24, 2020

(51) Int. Cl. G16H 50/20; G16H 10/60; G16H 70/60; G16H 70/40; G06F 16/23

(52) U.S. Cl. CPC G16H 50/20; G16H 10/60; G06F 16/2379; G16H 70/40; G16H 70/60

(57) ABSTRACT

The present disclosure provides computed-implemented method and computing device for predicting cancer. The computing device: retrieves an electronic medical record of a user from a database; transform the electronic medical record into a matrix; and determine a cancer prediction result corresponding to the matrix according to a cancer prediction model.

### TECHNICAL FIELD

The present disclosure relates to a computer-implemented method and a computing device thereof for predicting cancer of a user.

### BACKGROUND

Cancer involves abnormal cell growth, which has the potential to invade or spread to other parts of the human body. In the conventional diagnosis, determination of cancer usually depends on current medical images (e.g., x-ray images or x-ray computed tomography images) of the person and analyzed by an experienced doctor. However, there is still a need to improve accuracy of determination of cancer.

### SUMMARY

Some embodiments of the present disclosure provide a computer-implemented method for predicting cancer. The computer-implemented method includes: retrieving an electronic medical record of a user from a database; transforming the electronic medical record into a matrix; and determining a cancer prediction result corresponding to the matrix according to a cancer prediction model.

Some embodiments of the present disclosure provide a computer-implemented method for generating a caner prediction model. The computer-implemented method includes: retrieving a plurality of training data, wherein each training data includes an electronic medical record and a cancer result corresponding to the electronic medical record; transforming the electronic medical record into a matrix for each training data; and generating a cancer prediction model according to a machine learning scheme with the plurality of training data, wherein the matrix of each training data is used as training input data and the cancer result corresponding to the matrix is used as training output data.

Some embodiments of the present disclosure provide a computing device for predicting cancer. The computing device includes a processor and a storing unit. The storing unit stores a program that, when being executed, cause the processor to: retrieve an electronic medical record of a user from a database; transform the electronic medical record into a matrix; and determine a cancer prediction result corresponding to the matrix according to a cancer prediction model.

The present disclosure is described in detail in the following sections. Additional features and advantages of the disclosure will be described hereinafter and form the subject of the claims of the disclosure. It should be appreciated by those skilled in the art that the conception and specific embodiment disclosed may be readily utilized as a basis for modifying or designing other structures or processes for carrying out the same purposes of the present disclosure. It should also be realized by those skilled in the art that such equivalent constructions do not depart from the spirit and scope of the disclosure as set forth in the appended claims.

### BRIEF DESCRIPTION OF THE DRAWINGS

Aspects of the present disclosure are best understood from the following detailed description and figures. It is noted that, in accordance with the standard practice in the industry, various features are not drawn to scale. In fact, the dimensions of the various features may be arbitrarily increased or reduced for clarity of discussion.

A more complete understanding of the present disclosure may be derived by referring to the detailed description and claims when considered in connection with the Figures, where like reference numbers refer to similar elements throughout the Figures.

FIG. 1A is a block diagram of a computing device according to some embodiments of the present disclosure.

FIG. 1B is a block diagram of a computing device according to some embodiments of the present disclosure.

FIG. 1C is a schematic view of predicting cancer according to some embodiments of the present disclosure.

FIG. 2A is a schematic view of a matrix transformed from an electronic medical record according to some embodiments of the present disclosure.

FIG. 2B is a schematic view of a matrix transformed from an electronic medical record according to some embodiments of the present disclosure.

FIG. 3A is a schematic view of a matrix transformed from an electronic medical record according to some embodiments of the present disclosure.

FIG. 3B is a schematic view of a matrix transformed from an electronic medical record according to some embodiments of the present disclosure.

FIG. 4 is a schematic view of a matrix transformed from an electronic medical record according to some embodiments of the present disclosure.

FIG. 5 is a flowchart diagram of a computer-implemented method according to some embodiments of the present disclosure.

FIG. 6 is a flowchart diagram of a computer-implemented method according to some embodiments of the present disclosure.

### DETAILED DESCRIPTION

Embodiments, or examples, of the disclosure illustrated in the drawings are now described using specific language. It shall be understood that no limitation of the scope of the disclosure is hereby intended. Any alteration or modification of the described embodiments, and any further applications of principles described in this document, are to be considered as normally occurring to one of ordinary skill in the art to which the disclosure relates. Reference numerals may be repeated throughout the embodiments, but this does not necessarily mean that feature(s) of one embodiment apply to another embodiment, even if they share the same reference numeral.

It shall be understood that, although the terms first, second, third, etc. may be used herein to describe various elements, components, regions, layers or sections, these elements, components, regions, layers or sections are not limited by these terms. Rather, these terms are merely used to distinguish one element, component, region, layer or section from another element, component, region, layer or section. Thus, a first element, component, region, layer or section discussed below could be termed a second element, component, region, layer or section without departing from the teachings of the present inventive concept.

The terminology used herein is for the purpose of describing particular example embodiments only and is not intended to be limited to the present inventive concept. As used herein, the singular forms “a,” “an” and “the” are intended to include the plural forms as well, unless the context clearly indicates otherwise. It shall be further understood that the terms “comprises” and “comprising,” when used in this specification, point out the presence of stated features, integers, steps, operations, elements, or components, but do not preclude the presence or addition of one or more other features, integers, steps, operations, elements, components, or groups thereof. As used herein, a “user” can be defined, without limitation, to include a person/subject whose electronic medical records are suitably stored and/or processed and/or configured in accordance with and operative with various embodiments as described herein.

Cancers are considered as deadly diseases. However, the determinations or even the predictions of cancers are still inaccurate. Therefore, there is still a need for developing new methods and devices for precisely determining or predicting cancers.

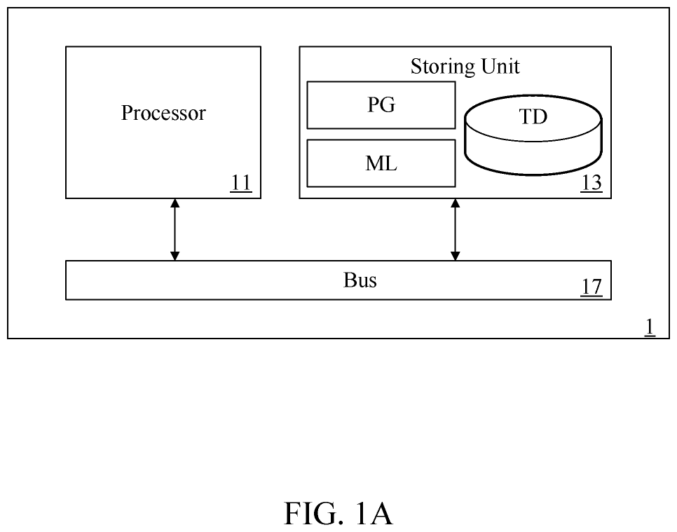

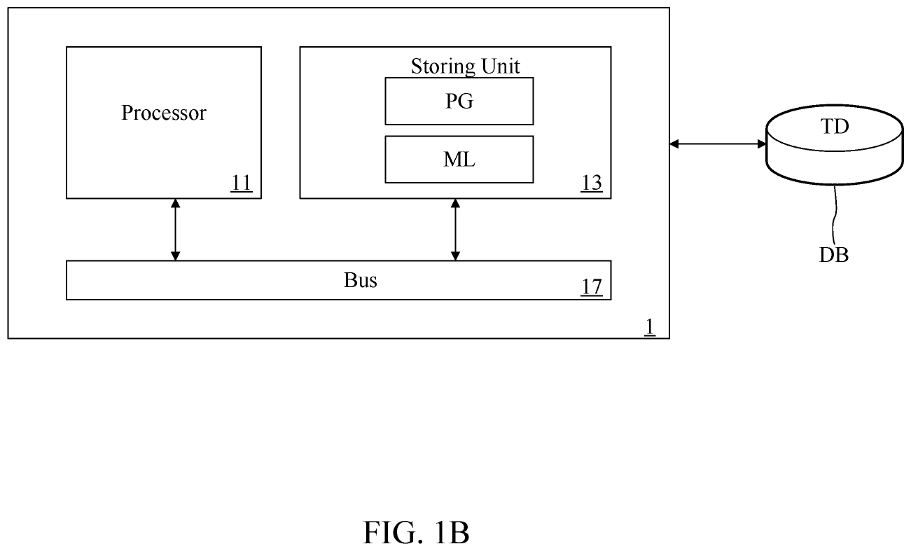

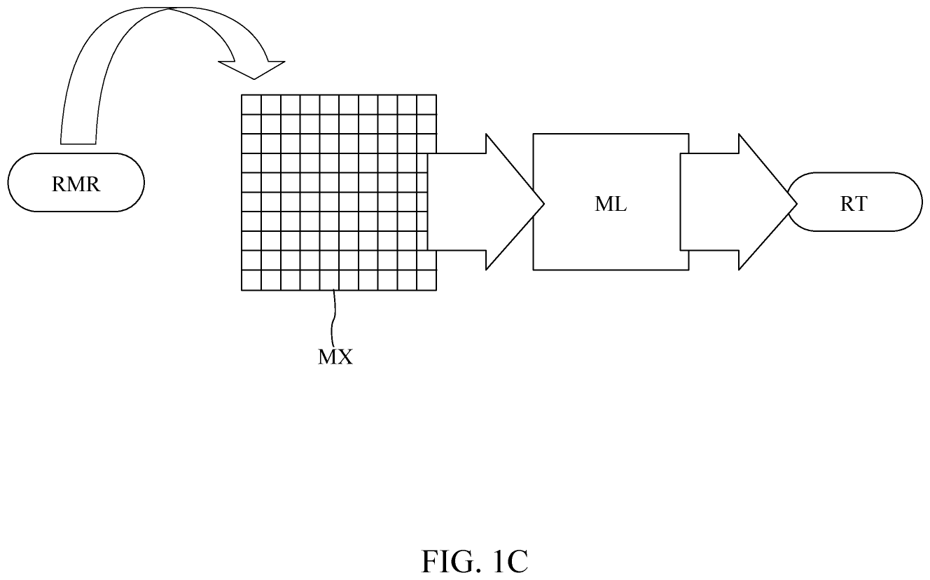

FIG. 1A illustrates a block diagram of a computing device 1 according to some embodiments of the present disclosure. The computing device 1 includes a processor 11 and a storing unit 13. The processor 11 and the storing unit 13 are electrically coupled through a communication bus 17.

The communication bus 17 may allow the processor 11 to execute a program PG stored in the storing unit 13. When executed, the program PG may generate one or more interrupts (e.g., software-interrupt) to cause the processor 11 to perform functions of the program PG for generating and utilizing cancer prediction model. The functions of the program PG will be further described hereinafter.

In some embodiments, a cancer prediction model ML may include a machine learning model which is generated according to a machine learning scheme with a plurality of training data TD. Particularly, in these embodiments, because the cancer prediction model ML may be used to receive data of user and output cancer prediction result for the user, some data of users and corresponding cancer results of these users may be used as the training data TD for training (i.e., generating) the cancer prediction model ML.

In some embodiments, the training data TD may include: (1) data of users; and (2) cancer results corresponding to these users. In detail, each of data of users may include an electronic medical record. The electronic medical record may include non-image data (e.g., text data) associated with anamnesis of corresponding user. Each of the cancer results may include an indicator which is used to indicate positive of cancer diagnosis or negative of cancer diagnosis.

It should be noted that, in some embodiments, the training data may be stored in an internal database (e.g., database of the storing unit 13 shown in FIG. 1A). In some implementations, the training data TD may be stored in an external database (e.g., database DB of an external storage or a cloud storage shown in FIG. 1B).

Then, when being executed, the program PG causes processor 11 to retrieve the electronic medical records of the training data TD from the database and to transform the electronic medical records into matrices. Next, the program PG causes processor 11 to generate (i.e., to train) the cancer prediction model ML according to: (1) the matrices transformed from the electronic medical records of the training data TD; and (2) cancer results corresponding to the electronic medical records.

Specifically, the matrices, which are transformed from the electronic medical records, may be used as training input data during the training stage. The cancer results corresponding to the electronic medical records may be used as training output data during the training stage. After the processor 11 generates the cancer prediction model ML, the storing unit 13 may store the cancer prediction model ML for later use.

It should be note that, in some embodiments, a Convolutional Neural Network (CNN) algorithm that is capable of building a model for predicting a result based on the training data is introduced for generating the cancer prediction model ML.

In particular, in the implementation (e.g., program code) of the CNN algorithm for training the cancer prediction model ML, there may be a training function (e.g., a function of the program code) for training the cancer prediction model ML. During the training of the cancer prediction model ML, the training function may include a section (e.g., part of the function) for receiving the training data TD.

Further, matrices, which are transformed from the electronic medical records, may be used as training input data. Cancer results corresponding to the electronic medical records may be used as training output data. Next, the cancer prediction model ML may be trained after the training function is executed with a main function (e.g., a main part of the program code) of the implementation of the CNN algorithm.

After generating the cancer prediction model ML with the training data (i.e., the training data TD) according to the CNN algorithm, the cancer prediction model ML may be used for predicting a result of cancer for a user.

Please refer to FIG. 1C. For example, when it is needed to determine or predict whether a user has cancer, the computing device 1 retrieves an electronic medical record RMR of the user from the database. Then, the computing device 1 transforms the electronic medical record RMR to a matrix MX. Next, the computing device 1 inputs the matrix MX into the cancer prediction model ML for outputting a cancer prediction result RT for the user.

In some embodiments, the cancer prediction result RT may include an indicator of negative or positive. If the cancer prediction result RT is negative, it means that the user may not have cancer. On the other hand, if the cancer prediction result RT is positive, it means that the user may have cancer.

In some embodiments, the cancer prediction result RT may include an indicator of probability. If the probability is not greater than a numerical threshold (e.g., in some certain nonlimiting embodiment, 0.4), it means that the user may not have cancer. On the other hand, if the probability is greater than the threshold, it means that the user may have cancer.

It should be noted that, in some embodiments, different models may be trained with different training data for predicting different types of cancer. Accordingly, in these embodiments, after training the cancer prediction model ML, the cancer prediction model ML may be used for predicting a type of cancer for a user.

In one embodiment, when the cancer prediction model ML is training with the training data related with lung cancer, the cancer prediction model ML can used for predicting lung cancer. In detail, the computing device 1 retrieves the electronic medical record RMR, which is related with lung cancer, of the user from the database. Then, the computing device 1 transforms the electronic medical record RMR to the matrix MX. Next, the computing device 1 inputs the matrix MX into the cancer prediction model ML for outputting a cancer prediction result RT for the user. The cancer prediction result RT may indicate whether the user has lung cancer.

In another embodiment, when the cancer prediction model ML is training with the training data related with skin cancer, the cancer prediction model ML can used for predicting skin cancer. In detail, the computing device 1 retrieves the electronic medical record RMR, which is related with skin cancer, of the user from the database. Then, the computing device 1 transforms the electronic medical record RMR to the matrix MX. Next, the computing device 1 inputs the matrix MX into the cancer prediction model ML for outputting a cancer prediction result RT for the user. The cancer prediction result RT may indicate whether the user has skin cancer.

For ease of understanding the mentioned technologies of the present disclosure, some examples of the above transformation between one electronic medical record and one matrix will be demonstrated hereinafter.

In some embodiments, an electronic medical record for being transformed into a matrix may include a plurality of International Classification of Diseases (ICD) data within a time period. Particularly, when the electronic medical record includes M number of ICD data within a time period including N number of time intervals, the electronic medical record may be transformed to an M by N matrix.

In detail, an element (m, n) of the M by N matrix includes a binary number, and in represents mₜₕ ICD data of the ICD data and n represents nₜₕ time interval of the time period. The element (m, n) is one value of the binary number when the electronic medical record indicates that the user is diagnosed with mₜₕ ICD data during nₜₕ time interval. The element (m, n) is another value of the binary number when the electronic medical record indicates that the user is not diagnosed with mₜₕ ICD data during nₜₕ time interval.

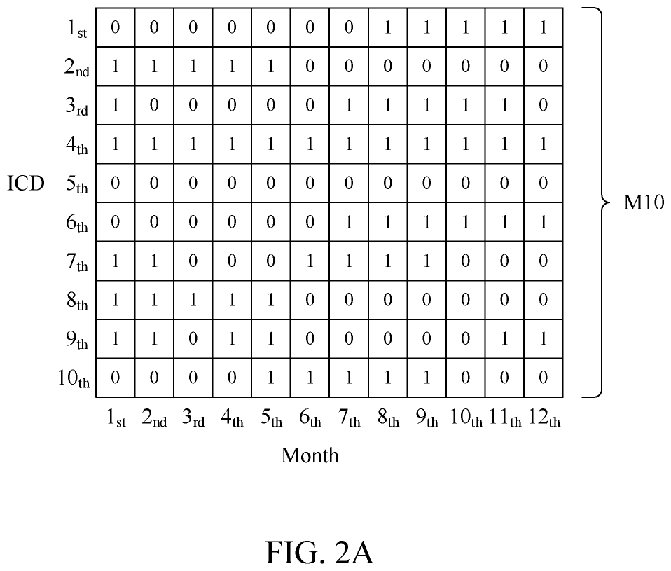

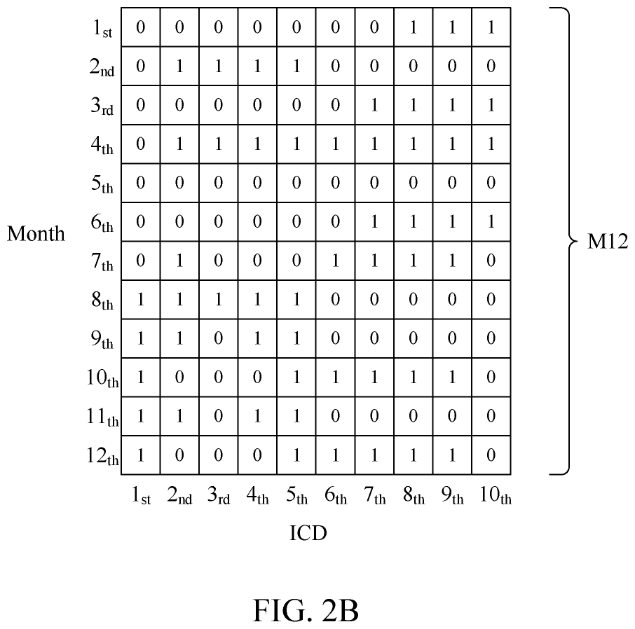

Please refer to FIG. 2A. In one embodiment, when the electronic medical record includes 10 ICD data within one year (i.e., time period) including 12 months (i.e., time interval), the computing device 1 parses the electronic medical record and transforms the electronic medical record into a 10 by 12 matrix M10.

The element (m, n) of the 10 by 12 matrix M10 is “1” of the binary number when the electronic medical record indicates that the user is diagnosed with mₜₕ ICD data during nₜₕ time interval. For instance, when 1ₛₜ ICD data correspond to diabetes data and the user is diagnosed with diabetes within the 8_{nd}, 9_{rd}, 10ₜₕ, 11ₜₕ and 12ₜₕ month of the year, the elements (1, 8), (1, 9), (1, 10), (1, 11) and (1, 12) of the 10 by 12 matrix M10 is “1”.

The element (m, n) of the 10 by 12 matrix M10 is “0” of the binary number when the electronic medical record indicates that the user is not diagnosed with mₜₕ ICD data during nₜₕ time interval. For instance, when 1ₛₜ ICD data correspond to diabetes data and the user is not diagnosed with diabetes within the 1ₛₜ, 2_{nd}, 3_{rd}, 4ₜₕ, 5ₜₕ, 6ₜₕ and 7ₜₕ month of the year, the element (1, 1), (1, 2), (1, 3), (1, 4), (1, 5), (1, 6) and (1, 7) of the 10 by 12 matrix M10 is “0”.

In some embodiments, when the electronic medical record includes M number of ICD data within the time period including N number of time intervals, the electronic medical record may be transformed to an N by M matrix.

In detail, an element (n, m) of the N by M matrix includes a binary number, and m represents mₜₕ ICD data of the ICD data and n represents nₜₕ time interval of the time period. The element (n, m) is one value of the binary number when the electronic medical record indicates that the user is diagnosed with mₜₕ ICD data during nₜₕ time interval. The element (n, m) is another value of the binary number when the electronic medical record indicates that the user is not diagnosed with mₜₕ ICD data during nₜₕ time interval.

Please refer to FIG. 2B. In one embodiment, when the electronic medical record includes 10 ICD data within one year (i.e., time period) including 12 months (i.e., time interval), the computing device 1 parses the electronic medical record and transforms the electronic medical record into a 12 by 10 matrix M12.

The element (n, m) of the 12 by 10 matrix M12 is “1” of the binary number when the electronic medical record indicates that the user is diagnosed with mₜₕ ICD data during nₜₕ time interval. For instance, when 1ₛₜ ICD data correspond to diabetes data and the user is diagnosed with diabetes within the 8_{nd}, 9_{rd}, 10ₜₕ, 11ₜₕ and 12ₜₕ month of the year, the elements (8, 1), (9, 1), (10, 1), (11, 1) and (12, 1) of the 12 by 10 matrix M12 is “1”.

The element (n, m) of the 12 by 10 matrix M12 is “0” of the binary number when the electronic medical record indicates that the user is not diagnosed with mₜₕ ICD data during nₜₕ time interval. For instance, when 1ₛₜ ICD data correspond to diabetes data and the user is not diagnosed with diabetes within the 1ₛₜ, 2_{nd}, 3_{rd}, 4ₜₕ, 5ₜₕ, 6ₜₕ and 7ₜₕ month of the year, the element (1, 1), (2, 1), (3, 1), (4, 1), (5, 1), (6, 1) and (7, 1) of the 12 by 10 matrix M12 is “0”.

In some embodiments, an electronic medical record for being transformed into a matrix may include a plurality of ICD data and a plurality of drug data within a time period. When the electronic medical record is includes “M1” number of ICD data and “M2” number of drug data within a time period including “N” number of time intervals, the electronic medical record may be transformed to an (M1+M2) by N matrix including an M1 by N sub-matrix and an M2 by N sub-matrix.

In detail, an element (m1, n1) of the M1 by N sub-matrix includes a binary number, m1 represents m1ₜₕ ICD data of the ICD data and n1 represents n1ₜₕ time interval of the time period. The element (m1, n1) is one value of the binary number when the electronic medical record indicates that the user is diagnosed with m1ₜₕ ICD data during n1ₜₕ time interval. The element (m1, n1) is another value of the binary number when the electronic medical record indicates that the user is not diagnosed with m1ₜₕ ICD data during n1ₜₕ time interval.

Further, an element (m2, n2) of the M2 by N sub-matrix includes 5 a binary number, m2 represents m2ₜₕ drug data of the drug data and n2 represents n2ₜₕ time interval of the time period. The element (m2, n2) is one value of the binary number when the electronic medical record indicates that the user has m2ₜₕ drug data during n2ₜₕ time interval (e.g., the user takes m2ₜₕ drug during n2ₜₕ time interval). The element (m2, n2) is another value of the binary number when the electronic medical record indicates that the user does not have m2ₜₕ drug data during n2ₜₕ time interval.

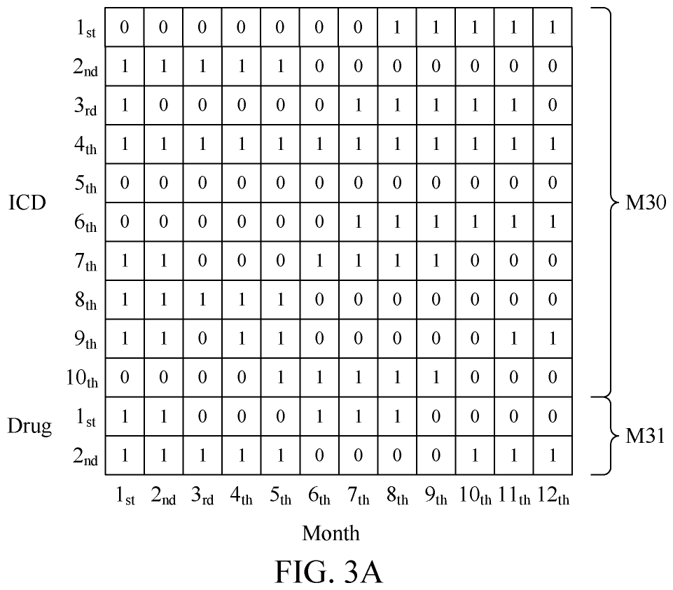

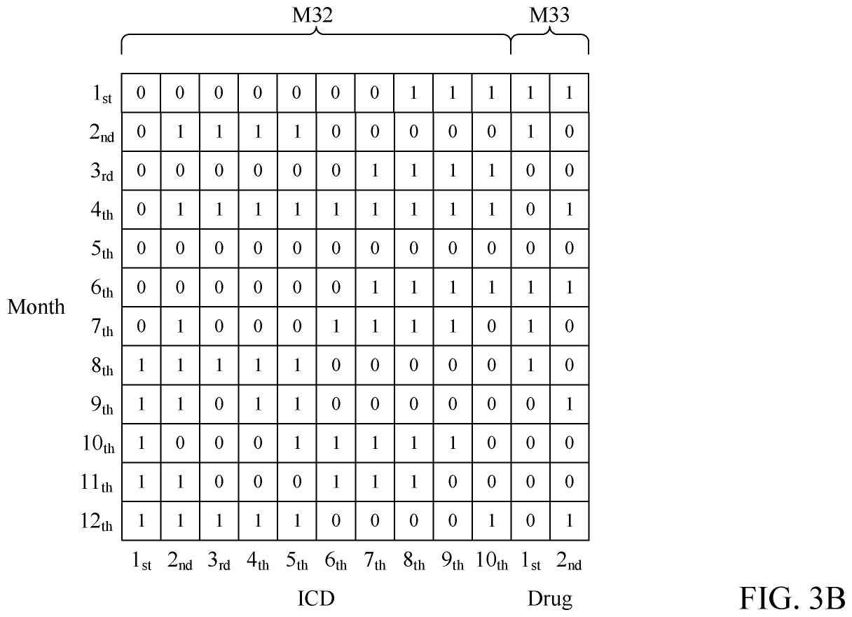

Please refer to FIG. 3A. For example, when the electronic medical record includes 10 ICD data and 2 drug data within one year (i.e., time period) including 12 months (i.e., time interval), the computing device 1 parses the electronic medical record and transforms the electronic medical record into a (10+2) by 12 matrix which includes a 10 by 12 sub-matrix M30 and a 2 by 12 sub-matrix M31.

The element (m1, n1) of the 10 by 12 sub-matrix M30 is “1” of the binary number when the electronic medical record indicates that the user is diagnosed with m1ₜₕ ICD data during n1ₜₕ time interval. For instance, when 1ₛₜ ICD data correspond to diabetes data and the user is diagnosed with diabetes within the 8_{nd}, 9_{rd}, 10ₜₕ, 11ₜₕ and 12ₜₕ month of the year, the elements (1, 8), (1, 9), (1, 10), (1, 11) and (1, 12) of the 10 by 12 matrix M30 is “1”.

The element (m1, n1) of the 10 by 12 sub-matrix M30 is “0” of the binary number when the electronic medical record indicates that the user is not diagnosed with m1ₜₕ ICD data during n1ₜₕ time interval. For instance, when 1ₛₜ ICD data correspond to diabetes data and the user is not diagnosed with diabetes within the 1ₛₜ, 2_{nd}, 3_{rd}, 4ₜₕ, 5ₜₕ, 6ₜₕ and 7ₜₕ month of the year, the element (1, 1), (1, 2), (1, 3), (1, 4), (1, 5), (1, 6) and (1, 7) of the 10 by 12 matrix M30 is “0”.

The element (m2, n2) of the 2 by 12 sub-matrix M31 is “1” of the binary number when the electronic medical record indicates that the user has m2ₜₕ drug data during n2ₜₕ time interval. For instance, when 1ₛₜ drug data correspond to penicillin data and the user is treated by penicillin within the 1ₛₜ, 2_{nd}, 6ₜₕ, 7ₜₕ and 8ₜₕ month of the year, the element (1, 1), (1, 2), (1, 6), (1, 7) and (1, 8) of the 2 by 12 matrix M31 is “1”.

The element (m2, n2) of the 2 by 12 sub-matrix M31 is “0” of the binary number when the electronic medical record indicates that the user does not have m2ₜₕ drug data during n2ₜₕ time interval. For instance, when 1ₛₜ drug data correspond to penicillin data and the user does not have any record of being treated by penicillin within the 3_{rd}, 4ₜₕ, 5ₜₕ, 9ₜₕ, 10ₜₕ, 11ₜₕ and 12ₜₕ month of the year, the element (1, 3), (1, 4), (1, 5), (1, 9), (1, 10), (1, 11) and (1, 12) of the 2 by 12 matrix M31 is “0”.

In some embodiments, when the electronic medical record includes “M1” number of ICD data and “M2” number of drug data within the time period including “N” number of time intervals, the electronic medical record may be transformed to an N by (M1+M2) matrix including an N by M1 sub-matrix and an N by M2 sub-matrix.

In detail, an element (n1, m1) of the N by M1 sub-matrix includes a binary number, m1 represents m1ₜₕ ICD data of the ICD data and n1 represents n1ₜₕ time interval of the time period. The element (n1, m1) is one value of the binary number when the electronic medical record indicates that the user is diagnosed with m1ₜₕ ICD data during n1ₜₕ time interval. The element (n1, m1) is another value of the binary number when the electronic medical record indicates that the user is not diagnosed with m1ₜₕ ICD data during n1ₜₕ time interval.

Further, an element (n2, m2) of the N by M2 sub-matrix includes a binary number, m2 represents m2ₜₕ drug data of the drug data and n2 represents n2ₜₕ time interval of the time period. The element (n2, m2) is one value of the binary number when the electronic medical record indicates that is the user has m2ₜₕ drug data during n2ₜₕ time interval (e.g., the user takes m2ₜₕ drug during n2ₜₕ time interval). The element (n2, m2) is another value of the binary number when the electronic medical record indicates that the user does not have m2ₜₕ drug data during n2ₜₕ time interval.

Please refer to FIG. 3B. In one embodiment, when the electronic medical record includes 10 ICD data and 2 drug data within one year (i.e., time period) including 12 months (i.e., time interval), the computing device 1 parses the electronic medical record and transforms the electronic medical record into a 12 by (10+2) matrix which includes a 12 by 10 sub-matrix M32 and a 12 by 2 sub-matrix M33.

The element (n1, m1) of the 12 by 10 sub-matrix M32 is “1” of the binary number when the electronic medical record indicates that the user is diagnosed with m1ₜₕ ICD data during n1ₜₕ time interval. For instance, when 1ₛₜ ICD data correspond to diabetes data and the user is diagnosed with diabetes within the 8_{nd}, 9_{rd}, 10ₜₕ, 11ₜₕ and 12ₜₕ month of the year, the elements (8, 1), (9, 1), (10, 1), (11, 1) and (12, 1) of the 12 by 10 matrix M32 is “1”.

The element (n1, m1) of the 12 by 10 sub-matrix M32 is “0” of the binary number when the electronic medical record indicates that the user is not diagnosed with m1ₜₕ ICD data during n1ₜₕ time interval. For instance, when 1ₛₜ ICD data correspond to diabetes and the user is not diagnosed with diabetes within the 1ₛₜ, 2_{nd}, 3_{rd}, 4ₜₕ, 5ₜₕ, 6ₜₕ and 7ₜₕ month of the year, the element (1, 1), (2, 1), (3, 1), (4, 1), (5, 1), (6, 1) and (7, 1) of the 12 by 10 matrix M32 is “0”.

The element (n2, m2) of the 12 by 2 sub-matrix M33 is “1” of the binary number when the electronic medical record indicates that the user has m2ₜₕ drug data during n2ₜₕ time interval. For instance, when 1ₛₜ drug data correspond to penicillin data and the user is treated by penicillin within the 1ₛₜ, 2_{nd}, 6ₜₕ, 7ₜₕ and 8ₜₕ month of the year, the element (1, 1), (2, 1), (6, 1), (7, 1) and (8, 1) of the 12 by 2 matrix M33 is “1”.

The element (n2, m2) of the 12 by 2 sub-matrix M33 is “0” of the binary number when the electronic medical record indicates that the user does not have m2ₜₕ drug data during n2ₜₕ time interval. For instance, when 1ₛₜ drug data correspond to penicillin data and the user does not have any record of being treated by penicillin within the 3_{rd}, 4ₜₕ, 5ₜₕ, 9ₜₕ, 10ₜₕ, 11ₜₕ and 12ₜₕ month of the year, the element (3, 1), (4, 1), (5, 1), (9, 1), (10, 1), (11, 1) and (12, 1) of the 12 by 2 matrix M33 is “0”.

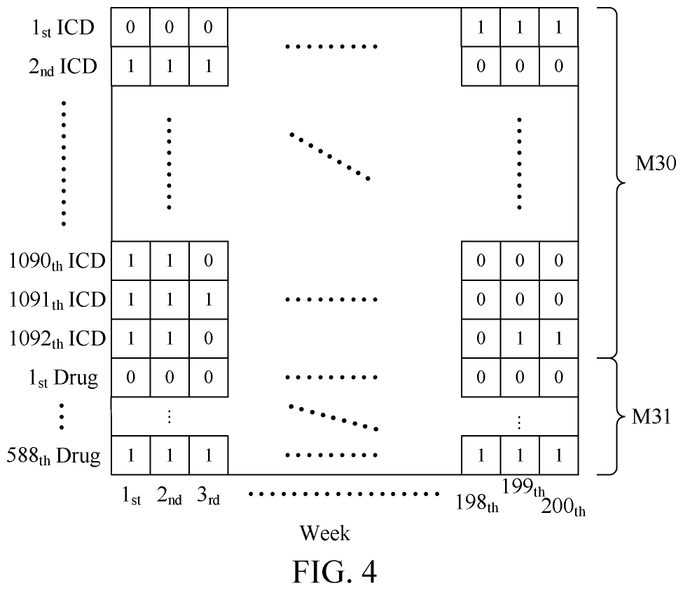

In some embodiments, the ICD data correspond to ICD Ninth Revision Clinical Modification (ICD-9-CM) which includes 1092 codes for different diseases associated with cancer. The drug data correspond Anatomical Therapeutic Chemical (ATC) code which includes 588 codes for different drugs associated with cancer. The time period includes 200 weeks. Accordingly, as shown in FIG. 4, an electronic medical record may be transformed into a (1092+588) by 200 matrix which includes a 1092 by 200 sub-matrix M30 and a 588 by 200 sub-matrix M31. In some implementations, the ICD data may correspond to ICD Tenth Revision Clinical Modification (ICD-10-CM) which includes 1878 codes for different diseases associated with cancer.

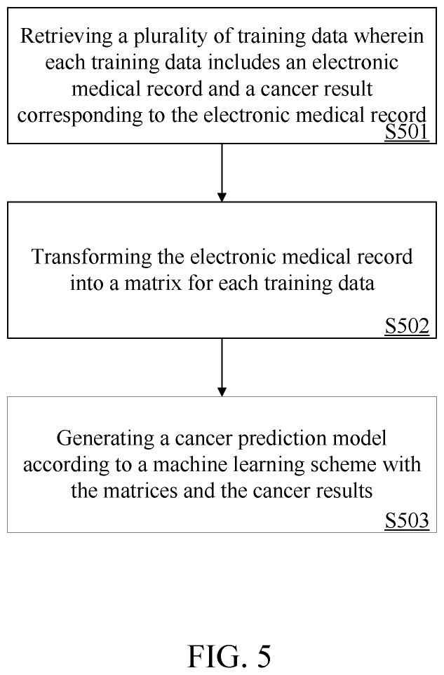

Some embodiments of the present disclosure include a computer-implemented method for generating a cancer prediction model, and a flowchart diagram thereof is shown in FIG. 5. The computer-implemented method of some embodiments is for use in a computing device (e.g., the computing device of the aforesaid embodiments). Detailed steps of the computer-implemented method are described below.

Step S501 is executed, by the computing device, to retrieve a plurality of training data. Each training data may include an electronic medical record and a cancer result corresponding to the electronic medical record. Step S502 is executed, by the computing device, to transform the electronic medical record into a matrix for each training data.

Step S503 is executed, by the computing device, to generate a cancer prediction model according to a machine learning scheme with the matrices and the cancer results of the training data. The matrix of each training data may be used as training input data and the cancer result corresponding to the matrix may be used as training output data. In some implementations, the cancer prediction model may be generated according to a CNN algorithm that is capable of building model for predicting a result based on the training data.

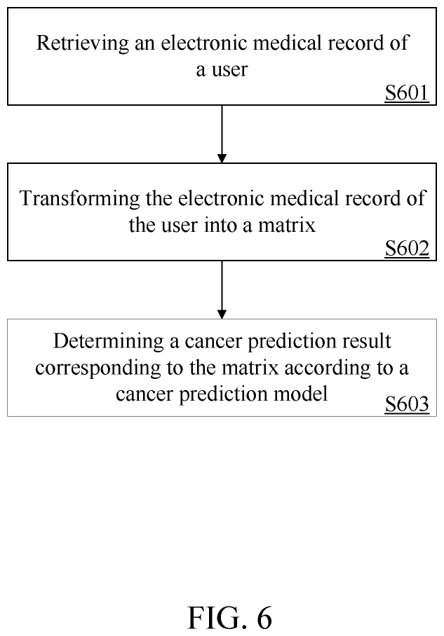

Some embodiments of the present disclosure include a computer-implemented method for predicting cancer, and a flowchart diagram thereof is shown in FIG. 6. The computer-implemented method of some embodiments is for use in a computing device (e.g., the computing device of the aforesaid embodiments). Detailed steps of the computer-implemented method are described below.

Step S601 is executed, by the computing device, to retrieve an electronic medical record of a user from a database. Step S602 is executed, by the computing device, to transform the electronic medical record into a matrix. Step S603 is executed, by the computing device, to determine a cancer prediction result corresponding to the matrix according to a cancer prediction model.

Different from conventional diagnostic method in which an experienced doctor needs to use image data (e.g., x-ray images or x-ray computed tomography images) to determine whether a user has a cancer, non-image data (i.e., the electronic medical records which include text data) and machine learning scheme are introduced in the present disclosure for predicting cancer more precisely.

It shall be particularly appreciated that the processors mentioned in the above embodiments may be a central processing unit (CPU), other hardware circuit elements capable of executing relevant instructions, or combination of computing circuits that are well-known by those skilled in the art based on the above disclosures.

Moreover, the storing units mentioned in the above embodiments may include memories, such as ROM, RAM, etc., or storing device, such as flash memory, HDD, SSD, etc., for storing data. Further, the communication buses mentioned in the above embodiments may include a communication interface for transferring data between the elements, such as the processor, the storing unit, the sensor and the alert element, and may include electrical bus interface, optical bus interface or even wireless bus interface. However, such description is not intended to limit the hardware implementation embodiments of the present disclosure.

Although the present disclosure and its advantages have been described in detail, it should be understood that various changes, substitutions and alterations can be made herein without departing from the spirit and scope of the disclosure as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A computer-implemented method for predicting cancer, comprising:
retrieving an electronic medical record of a user from a database;
transforming the electronic medical record into a matrix; and
determining a cancer prediction result corresponding to the matrix according to a cancer prediction model.

2. The computer-implemented method of claim 1, wherein the electronic medical record includes at least one International Classification of Diseases (ICD) data within a time period.

3. The computer-implemented method of claim 2, wherein transforming the electronic medical record into the matrix further comprises:
transforming the at least one ICD data within the time period to the matrix, wherein the matrix includes an M by N matrix, an element (m, n) of the M by N matrix includes a binary number, M represents a number of the at least one ICD data, N represents a number of time intervals of the time period, in represents mₜₕ ICD data of the at least one ICD data and n represents nₜₕ time interval of the time period;
wherein,
the element (m, n) is one value of the binary number when the electronic medical record indicates that the user is diagnosed with mₜₕ ICD data during nₜₕ time interval; and
the element (m, n) is another value of the binary number when the electronic medical record indicates that the user is not diagnosed with mₜₕ ICD data during nₜₕ time interval.

4. The computer-implemented method of claim 2, wherein the electronic medical record further includes at least one drug data within the time period.

5. The computer-implemented method of claim 4, wherein transforming the electronic medical record into the matrix further comprises:
transforming the at least one ICD data and the at least one drug data within the time period to the matrix, wherein the matrix includes an M1 by N sub-matrix and a M2 by N sub-matrix, M1 represents a number of the at least one ICD data, M2 represents a number of the at least one drug data, N represents a number of time intervals of the time period,
wherein an element (m1, n1) of the M1 by N sub-matrix includes a binary number while m1 represents m1ₜₕ ICD data of the at least one ICD data and n1 represents n1ₜₕ time interval of the time period,
wherein,
the element (m1, n1) is one value of the binary number when the electronic medical record indicates that the user is diagnosed with m1ₜₕ ICD data during n1ₜₕ time interval; and
the element (m1, n1) is another value of the binary number when the electronic medical record indicates that the user is not diagnosed with m1ₜₕ ICD data during n1ₜₕ time interval,
wherein an element (m2, n2) of the M2 by N sub-matrix includes a binary number while m2 represents m2ₜₕ drug data of the at least one drug data and n2 represents n2ₜₕ time interval of the time period,
wherein,
the element (m2, n2) is one value of the binary number when the electronic medical record indicates that the user has m2ₜₕ drug data during n2ₜₕ time interval; and
the element (m2, n2) is another value of the binary number when the electronic medical record indicates that the user does not have m2ₜₕ drug data during n2ₜₕ time interval.

6. The computer-implemented method of claim 2, wherein the at least one ICD data corresponds to ICD, Ninth Revision, Clinical Modification (ICD-9-CM) or ICD, Tenth Revision, Clinical Modification (ICD-10-CM).

7. The computer-implemented method of claim 1, further comprising:
is generating the cancer prediction model according to a machine learning scheme with a plurality of training data, wherein each training data includes a training input data and a training output data, the training input data includes a training matrix and the training output data includes a training cancer result corresponding to the training matrix.

8. The computer-implemented method of claim 7, further comprising:
transforming a training electronic medical record into the training matrix for each training data.

9. The computer-implemented method of claim 1, wherein the electronic medical record includes text data.

10. A computer-implemented method for generating a caner prediction model, comprising:
retrieving a plurality of training data, wherein each training data includes an electronic medical record and a cancer result corresponding to the electronic medical record;
transforming the electronic medical record into a matrix for each training data; and
generating a cancer prediction model according to a machine learning scheme with the plurality of training data, wherein the matrix of each training data is used as training input data and the cancer result corresponding to the matrix is used as training output data.

11. A computing device for predicting cancer, comprising:
a processor; and
a storing unit including a program that, when being executed, causes the processor to:
retrieve an electronic medical record of a user;
transform the electronic medical record into a matrix; and
determine a cancer prediction result corresponding to the matrix according to a cancer prediction model.

12. The computing device of claim 11, wherein the electronic medical record associated with the storing unit containing the program includes at least one International Classification of Diseases (ICD) data within a time period.

13. The computing device of claim 12, wherein the program, when being executed, further causes the processor to:
transform the at least one ICD data within the time period to the matrix, wherein the matrix includes an M by N matrix, an element (m, n) of the M by N matrix includes a binary number, M represents a number of the at least one ICD data, N represents a number of time intervals of the time period, in represents mₜₕ ICD data of the at least one ICD data and n represents nₜₕ time interval of the time period;
wherein,
the element (m, n) is one value of the binary number when the electronic medical record indicates that the user is diagnosed with mₜₕ ICD data during nₜₕ time interval; and
the element (m, n) is another value of the binary number when the electronic medical record indicates that the user is not diagnosed with mₜₕ ICD data during nₜₕ time interval.

14. The computing device of claim 12, wherein the electronic medical record further includes at least one drug data within the time period.

15. The computing device of claim 14, wherein the program, when being executed, further causes the processor to:
transform the at least one ICD data and the at least one drug data within the time period to the matrix, wherein the matrix includes an M1 by N sub-matrix and a M2 by N sub-matrix, M1 represents a number of the at least one ICD data, M2 represents a number of the at least one drug data, N represents a number of time intervals of the time period,
wherein an element (m1, n1) of the M1 by N sub-matrix includes a binary number while m1 represents m1ₜₕ ICD data of the at least one ICD data and n1 represents n1ₜₕ time interval of the time period,
wherein,
the element (m1, n1) is one value of the binary number when the electronic medical record indicates that the user is diagnosed with m1ₜₕ ICD data during n1ₜₕ time interval; and
the element (m1, n1) is another value of the binary number when the is electronic medical record indicates that the user is not diagnosed with m1ₜₕ ICD data during n1ₜₕ time interval,
wherein an element (m2, n2) of the M2 by N sub-matrix includes a binary number while m2 represents m2ₜₕ drug data of the at least one drug data and n2 represents n2ₜₕ time interval of the time period,
wherein,
the element (m2, n2) is one value of the binary number when the electronic medical record indicates that the user has m2ₜₕ drug data during n2ₜₕ time interval; and
the element (m2, n2) is another value of the binary number when the electronic medical record indicates that the user does not have m2ₜₕ drug data during n2ₜₕ time interval.

16. The computing device of claim 12, wherein the at least one ICD data corresponds to ICD, Ninth Revision, Clinical Modification (ICD-9-CM) or ICD, Tenth Revision, Clinical Modification (ICD-10-CM).

17. The computing device of claim 12, wherein the program, when being executed, further causes the processor to:
generate the cancer prediction model according to a machine learning scheme with a plurality of training data, wherein each training data includes a training input data and a training output data, the training input data includes a training matrix and the training output data includes a training cancer result corresponding to the training matrix.

18. The computing device of claim 17, wherein the program, when being executed, further causes the processor to:
transform a training electronic medical record into the training matrix for each training data.
